(19) United States
(12) Patent Application Publication
(10) Pub. No.: US 2024/0058157 A1
(43) Pub. Date: Feb. 22, 2024

(54) FLUID COLLECTION DEVICES AND SYSTEMS INCLUDING AN ADHESIVE SECUREMENT FEATURE, AND METHODS OF USE

(71) Applicant: PUREWICK CORPORATION, Covington, GA (US)

(72) Inventor: Kathleen Davis, Atlanta, GA (US)

(21) Appl. No.: 18/006,807

(22) PCT Filed: Aug. 4, 2021
(86) PCT No.: PCT/IB2021/057173

§ 371 Date: Jan. 25, 2023

Related U.S. Application Data
(60) Provisional application No. 63/061,244, filed on Aug. 5, 2020.

(51) Int. Cl. A61F 5/44; A61F 5/451

(52) U.S. Cl. CPC A61F 5/4404; A61F 5/451

(57) ABSTRACT

Example fluid collection devices and methods of use arc described. The fluid collection devices include a fluid impermeable barrier, a fluid permeable body, and two arms. The fluid impermeable barrier at least partially defines a chamber, and an opening configured to be positioned adjacent to a urethra. The fluid permeable body is positioned at least partially within the chamber to extend across at least a portion of the opening and configured to wick fluid away from the opening. The arms include an adhesive surface oriented towards the fluid impermeable barrier. The arms arc positioned on the fluid impermeable barrier to attach to at least one of an abdominal region, a pubic region, or a pelvic region of the user when the opening of the fluid impermeable barrier is positioned at least proximate to the urethra of the user.

### CROSS-REFERENCE TO RELATED APPLICATIONS

This application claims priority to U.S. Provisional Patent Application No. 63/061,244 filed on Aug. 5, 2020, the disclosure of which is incorporated herein, in its entirety, by this reference.

### BACKGROUND

An individual may have limited or impaired mobility such that typical urination processes are challenging or impossible. For example, the individual may have surgery or a disability that impairs mobility. In another example, the individual may have restricted travel conditions such as those experienced by pilots, drivers, and workers in hazardous areas. Additionally, fluid collection from the individual may be needed for monitoring purposes or clinical testing.

Bed pans and urinary catheters, such as a Foley catheter, can be used to address some of these circumstances. However, bed pans and urinary catheters have several problems associated therewith. For example, bed pans can be prone to discomfort, pressure ulcers spills, and other hygiene issues. Urinary catheters be can be uncomfortable, painful, and can cause urinary tract infections.

Thus, users and manufacturers of fluid collection devices continue to seek new and improved devices, systems, and methods to collect urine.

### SUMMARY

Embodiments disclosed herein are fluid collection devices, methods of assembling fluid collection devices, and methods of collecting fluids. In an embodiment, a fluid collection device includes a fluid impermeable barrier, a fluid permeable body, and one or more arms or wings. The fluid impermeable barrier at least partially defines a chamber, an opening extending longitudinally along the fluid impermeable barrier and configured to be positioned adjacent to a urethra, and an aperture configured to receive a conduit therethrough. The fluid permeable body is positioned at least partially within the chamber to extend across at least a portion of the opening and configured to wick fluid away from the opening. The one or more arms include an adhesive surface oriented towards the fluid impermeable barrier. The one or mom arms are positioned on the fluid collection device to attach to at least one of an abdominal region, a pubic region, or a pelvic region of the user when the opening of the fluid impermeable barrier is positioned at least proximate to the urethra of the user.

In an embodiment, a method of collecting fluids from a user includes positioning fluid permeable body of a fluid collection device to cover a urethra of a user. The method also includes applying one or more adhesive arms to at least one of a pubic region, a pelvic region, or an abdominal region of the user effective to secure the fluid collection device to the user. The one or more adhesive arms are secured to at least one of a fluid impermeable barrier or a conduit of the fluid collection device. The method also includes receiving fluids discharged from the urethra into a chamber of the fluid collection device.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is an isometric view of a fluid collection device having an adhesive securement feature, according to an embodiment.

FIG. 1B is a front view of a female user with the fluid collection device of FIG. 1A being worn by the female user.

FIG. 1C is a rear view of the fluid collection device of FIG. 1A.

FIG. 1D is a cross-sectional view of the fluid collection device of FIG. 1A taken along line 1-1.

FIG. 1E is an exploded view of the fluid collection device of FIG. 1A.

FIG. 1F is an isometric view of a fluid collection device having an adhesive securement feature attached thereto, according to an embodiment.

FIG. 1G is a front view of a female user with a fluid collection device being worn by the female user.

FIG. 2A is an isometric view of a fluid collection device having an adhesive securement feature attached to a conduit, according to an embodiment.

FIG. 2B is a front view of a female user with the fluid collection device of FIG. 2A being worn by the female user.

FIG. 2C is a front view of the fluid collection device of FIG. 2A.

FIG. 2D is a front view of a conduit of a fluid collection device, according to an embodiment.

FIG. 3 is a flow diagram of a method of assembling a fluid collection device, according to an embodiment.

FIG. 4 is a flow diagram of a method to collect fluid, according to an embodiment.

FIG. 5 is a block diagram of a system for fluid collection, according to an embodiment.

### DETAILED DESCRIPTION

Embodiments disclosed herein are fluid collection devices, methods of assembling fluid collection devices, and methods of collecting fluids. Fluid collection devices are used to collect fluids from a user, such as urine, vaginal discharge, penile discharge, reproductive fluids, blood, sweat, or other bodily fluids. Fluid collection devices described herein effectively and efficiently collect fluid from a user when the fluid collection device is held at least proximate to (e.g., adjacent to) the urethra of the user. Embodiments of fluid collection disclosed herein include one or more adhesive securement features configured to attach to the user to secure the fluid collection device in the desired position with the opening of the fluid collection device proximate to the urethra of the user. The one or more adhesive securement features may be positioned on at least one of the fluid impermeable barrier or the conduit of the fluid collection devices resulting in the technical effect of attaching the one or more adhesive securement features to the abdominal, pubic, or pelvic area of the user. Once attached to the user, the one or more adhesive securement features also may provide the technical effect of preventing dislodgement of the fluid collection device from the desired position against the user.

In an embodiment, a fluid collection device includes a fluid impermeable barrier, a fluid permeable body, and one or more arms each having an adhesive surface. The fluid impermeable barrier at least partially defines a chamber, an opening extending longitudinally along the fluid impermeable barrier and configured to be positioned adjacent to a female urethra, and an aperture configured to receive a conduit therethrough. The fluid collection devices described herein may be shaped and sized to be positioned over the urethra opening of a male having a buried penis or have a male urethra positioned therethrough (e.g., receive a penis therein). The fluid permeable body is positioned at least partially within the chamber to extend across at least a portion of the opening and configured to wick fluid away from the opening. The adhesive surface of each of the one or more arms or wings may include a material including an adhesive such as an acrylic adhesive, a silicone adhesive, a hydrogel adhesive, other adhesives appropriate for contacting the body of a user, or any combinations thereof.

Fluid collection devices described herein may be used in fluid collection systems and methods. The fluid collection systems may include a fluid collection device, a fluid storage container, and a portable vacuum source. Fluid (e.g., urine or other bodily fluids) collected in the fluid collection device may be removed from the fluid collection device via a conduit which protrudes into an interior region of the fluid collection device. For example, a first open end of the conduit may extend into the fluid collection device to a reservoir therein. The second open end of the conduit may extend into the fluid collection device or the portable vacuum source. The suction force may be introduced into the interior region of the fluid collection device via the first open end of the conduit responsive to a suction (e.g., vacuum) force applied at the second end of the conduit. The suction force may be applied to the second open end of the conduit by the portable vacuum source either directly or indirectly.

In some embodiments, the portable vacuum source may be disposed in or on the fluid collection device. In such embodiments, the conduit may extend from the fluid collection device and attach to the portable vacuum source at a first point therein. An additional conduit may attach to the portable vacuum source at a second point thereon and may extend out of the fluid collection device, and may attach to the fluid storage container. Accordingly, a vacuum (e.g., suction) may be drawn through the fluid collection device via the fluid storage container. Fluid, such as urine, may be drained from the fluid collection device using the portable vacuum source.

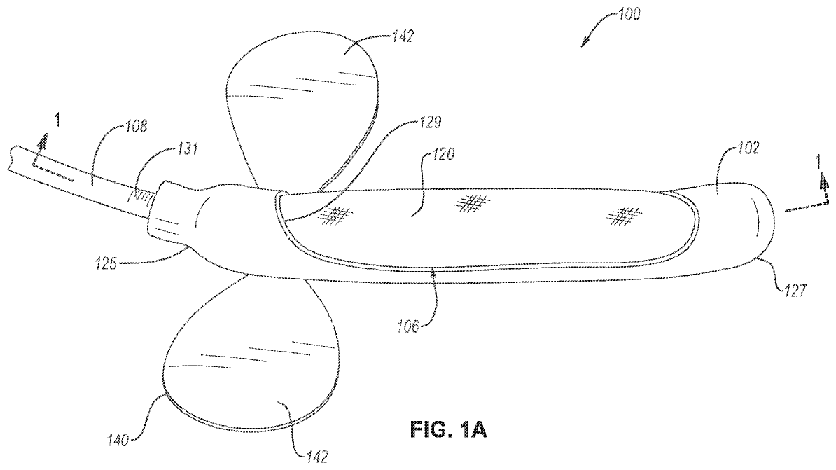

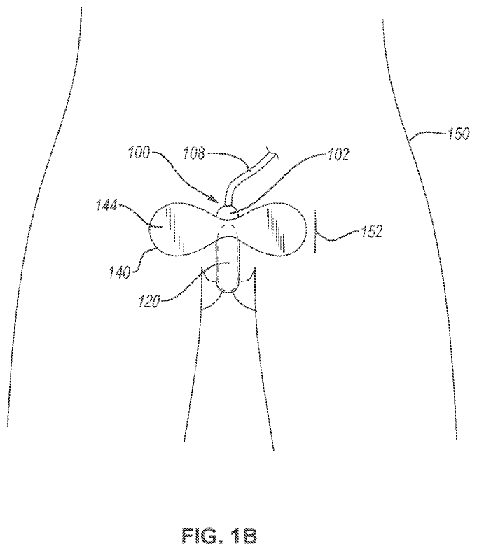

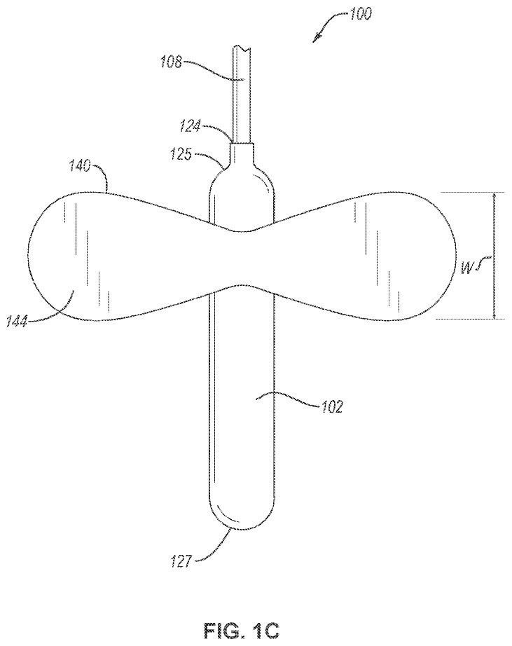

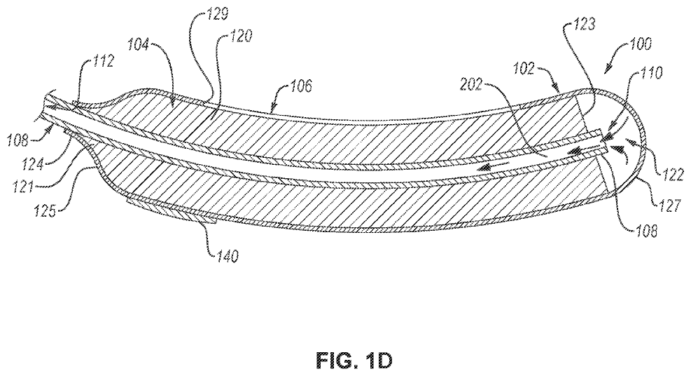

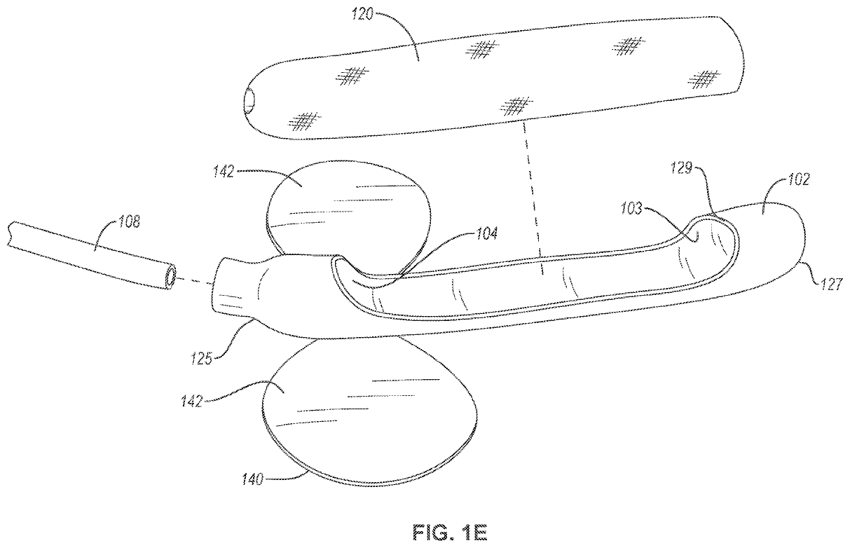

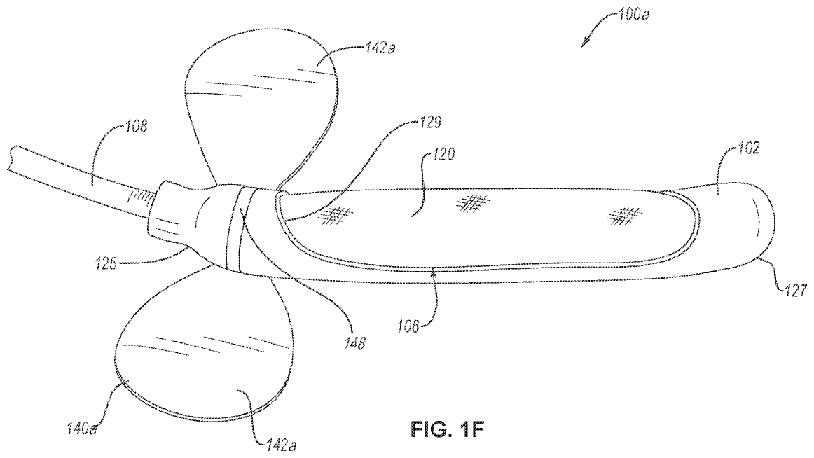

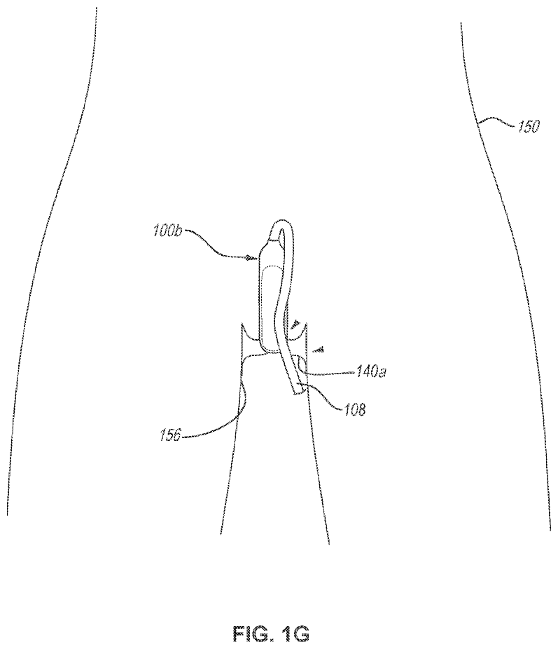

FIG. 1A is an isometric view of a fluid collection device 100 having an adhesive securement feature secured thereto, according to an embodiment. The fluid collection device 100 is an example of a fluid collection device 100 that is configured to receive fluids from a female or a male having a buried penis. The fluid collection device 100 includes a fluid impermeable barrier 102 having a first end region 125 and a second end region 127. The fluid impermeable barrier 102 at least partially defines a chamber 104 (e.g., interior region, shown in FIG. 1E) and includes an inward border or edge 129 defining an opening 106. The fluid impermeable harrier 102 is substantially cylindrical in shape between the first end region 125 and the second end region 127. In other embodiments, the fluid impermeable barrier 102 may include other shapes, such as one of more substantially planar surfaces, triangular, or other suitable shapes. The opening 106 is formed in and extends longitudinally through the fluid impermeable barrier 102, thereby enabling fluids to enter the chamber 104 from outside of the fluid collection device 100. The opening 106 may be configured to be positioned at least proximate to (e.g., adjacent to) the opening of a female urethra or over the opening of a urethra of a male having a buried penis.

The fluid collection device 100 may be positioned at least proximate to the opening of the urethra and urine may enter the interior region of the fluid collection device 100 via the opening 106. The fluid collection device 100 is configured to receive the fluids into the chamber 104 via the opening 106. For example, the opening 106 may exhibit an elongated shape that is configured to extend from a first location below the urethral opening (e.g., at or near the anus or the vaginal opening) to a second location above the urethral opening (e.g., at or near the clitoris or the pubic hair). The opening 106 may exhibit an elongated shape since the space between the legs of a female is relatively small when the legs of the female are closed, thereby only permitting the flow of the fluids along a path that corresponds to the elongated shape of the opening 106. For example, the opening may extend longitudinally along the fluid impermeable barrier. The opening 106 in the fluid impermeable barrier 102 may exhibit a width that is measured transverse to the longitudinal direction and may be at least about 10% of the circumference of the fluid collection device 100, such as about 25% to about 50%, about 40% to about 60%, about 50% to about 75%, about 65% to about 85%, or about 75% to about 100% of the circumference of the fluid collection device 100. The opening 106 may exhibit a width that is greater than 50%, of the circumference of the fluid collection device 100 since the vacuum (e.g., suction) through the conduit 108 pulls the fluid into the conduit 108. In some embodiments, the opening 106 may be vertically oriented (e.g., having a major axis parallel to the longitudinal axis of the device 100). In some embodiments, (not shown), the opening 106 may be horizontally oriented (e.g., having a major axis perpendicular to the longitudinal axis of the device 100). In some embodiments, the inward border or edge 129 of the fluid impermeable barrier 102 defines the opening 106. The edge 129 may include two opposing axed portions, the are portions following the outer circumference or periphery of the substantially cylindrical fluid impermeable barrier 102.

The fluid impermeable barrier 102 may also temporarily store the fluids in the chamber 104. For example, the fluid impermeable barrier 102 may be formed of any suitable fluid impermeable materials, such as a fluid impermeable polymer (e.g., silicone, polypropylene, polyethylene, polyethylene terephthalate, a polycarbonate, etc.), polyurethane films, thermoplastic elastomer (TPE), rubber, thermoplastic polyurethane, another suitable material, or combinations thereof. As such, the fluid impermeable barrier 102 substantially prevents the fluids from exiting the portions of the chamber 104 that are spaced from the opening 106. The fluid impermeable barrier 102 is flexible, thereby enabling the fluid collection device 100 to bend or curve when positioned against the body of a wearer. Example fluid impermeable barriers may include, but are not limited to, a fluid impermeable barrier including at least one of Versaflex CL 2000X TPE, Dynaflex G6713 TPE, or Silpuran 6000/05 A/B silicone.

In an embodiment, the fluid impermeable barrier 102 may be air permeable. In such an embodiment, the fluid impermeable barrier 102 may be formed of a hydrophobic material that defines a plurality of pores. In an embodiment, one or mom portions of at least the outer surface of the fluid impermeable barrier 102 may be formed from a soft and/or smooth material, thereby reducing chaffing. The fluid impermeable barrier 102 may include markings thereon, such as one or more markings to aid a user in aligning the device 100 on the wearer. For example, a line on the fluid impermeable barrier 102 (e.g., opposite the opening 106) may allow a healthcare professional to align the opening 106 over the urethra of the wearer. In examples, the markings may include one or more of alignment guide or an orientation indicator, such as a stripe or hashes. Such markings may be positioned to align the device 100 to one or more anatomical features such as a pubic bone, etc.

In an embodiment, fluid collection device 100 includes an adhesive securement feature secured or securable to the fluid impermeable barrier 102 and configured to attach the fluid collection device 100 to an individual. The one or more adhesive securement features may be positioned on at least one of the fluid impermeable barrier or the conduit of the fluid collection devices resulting in the technical effect of attaching the one or more adhesive securement features to the abdominal, pubic, or pelvic area of the user. Once attached to the user, the one or more adhesive securement features also may provide the technical effect of preventing dislodgement of the fluid collection device from the desired position against the user. For example, the fluid collection device 100 shown in FIG. 1A includes one or more (e.g., two) arms 140 or wings configured to attach or secure the fluid collection device 100 to an individual. The arms 140 may each include an adhesive surface 142 oriented towards the opening 106 or generally in the same direction as the opening 106 is oriented. The adhesive surface 142 may include an adhesive material, such as a glue, contact adhesive, epoxy, hydrogel adhesive, tape, an acrylic adhesive, a silicone adhesive, a hydrogel adhesive, other adhesives suitable for placement on the body of a user or fabric worn by the user, or any combination thereof. For example, the adhesive may include an acrylate (e.g., methacrylate or epoxy diacrylate) or any other adhesive suitable for use on bandages. The arms 140 also may include a removable cover secured to the adhesive layer 142 that may be removed prior to securing the adhesive layer to the user.

FIG. 1B is a front view of a female user 150 with the fluid collection device 100 secured to the female user 150. In use, the fluid permeable body 120 of the fluid collection device is positioned adjacent to a urethra of the user 150. The fluid permeable body 120 is disposed within a chamber 104 (shown in FIG. 1D) of the fluid impermeable barrier 102 of the fluid collection device 100 and is exposed to the urethra of the user 150 through the opening 106 in the fluid collection device 100. Fluids received in the chamber 104 of the fluid collection device 100 from the urethra may be removed through the conduit 108.

The adhesive surface 142 of each of the arms 140 may attach the fluid collection device 100 to the user 150, and the arms 140 also may include a non-adhesive surface 144 opposite or distal to the adhesive surface 142. The adhesive surface 142 may attach directly to the skin of the user 150 or may attach to the fabric worn by the user 150, such as undergarments. As shall be described in greater detail below, the arms 140 may be positioned at various areas on the fluid collection device 100 to secure the fluid collection device 100 directly to a predetermined area of the body of the user 150, such as the abdominal region, the pubic region, the pelvic region, the hypogastrium (or hypogastric) region, the legs (including thighs) of the user 150, or any combination thereof. In FIG. 1B, the arms 140 are positioned on the fluid collection device 100 to provide the technical effect of attaching the adhesive surface 142 of each of the arms to the pelvic region 152 of the user 150. The pelvic region 152 may include the area of the user 150 between the abdomen and the pubic bone or labia of the user 150.

Turning to FIG. 1C, which shows a rear view of a fluid collection device 100, in some embodiments, the two arms 140 are positioned on the fluid collection device 100 on a rear or back surface of the fluid impermeable barrier 102, generally distal to the opening 106. The two arms 140 may be positioned at various positions on the rear or back surface of the fluid impermeable barrier 102. For example, in the fluid collection device 100 shown in FIG. 1C, the two arms are positioned on rear or hack surface of the fluid impermeable barrier 102 proximate to the first end region 127 and less than about 5.1 cm from the aperture 124 to provide the technical effect of attaching the two arms 140 to the pelvis or lower abdomen of the user 150. In other embodiments, the arms 140 may be positioned elsewhere on the rear or back of the fluid impermeable barrier 102 effective to provide the technical effect of attaching the two arms 140 to other areas of the body of the user 150, as shall be shown below. In various embodiments, the arms 140 may be positioned on the rear or back surface less than about 15.2 cm from the aperture 124, less than about 12.7 cm from the aperture 124, less than about 10.2 cm from the aperture 124, less than about 7.6 cm from the aperture 124, less than about 5.1 cm from the aperture 124, less than about 2.5 cm from the aperture 124, less than about 1.3 cm from the aperture 124, more than about 2.5 cm from the aperture 124, more than about 5.1 cm from the aperture 124, more than about 7.6 cm from the aperture 124, more than about 10.2 cm from the aperture 124, more than about 12.7 cm from the aperture 124, more than about 15.2 cm from the aperture 124, more than about 17.8 cm from the aperture 124, more than about 20.3 cm from the aperture, about 1.3 cm to about 3.8 cm from the aperture 124, about 2.5 cm to about 5.1 cm from the aperture 124, about 5.1 cm to about 7.6 cm from the aperture 124, about 7.6 cm to about 10.2 cm from the aperture 124, about 10.2 cm to about 12.7 cm from the aperture 124, about 12.7 cm to about 15.2 cm from the aperture 124, about 15.2 cm to about 17.8 cm from the aperture 124, about 17.8 cm to about 20.3 cm from the aperture 124, about 20.3 cm to about 22.9 cm from the aperture 124, about 1.3 cm from the aperture 124, about 2.5 cm from the aperture 124, about 3.8 cm from the aperture 124, about 5.1 cm from the aperture 124, about 6.4 cm from the aperture 124, about 7.6 cm from the aperture 124, about 8.9 cm from the aperture 124, about 10.2 cm from the aperture 124, about 11.4 cm from the aperture 124, about 12.7 cm from the aperture 124, about 14 cm from the aperture 124, about 15.2 cm from the aperture 124, about 16.5 cm from the aperture 124, about 17.8 cm from the aperture 124, about 19.1 cm from the aperture 124, about 20.3 cm from the aperture 124, about 21.6 cm from the aperture 124, or about 22.9 cm from the aperture 124.

In some embodiments (not shown), the two arms 140 may be positioned on the fluid collection device on a front surface of the fluid impermeable barrier 102, such as on the first end region 125 between the opening 106 and the aperture 124 or on the second end region 127 between the opening and the distal end of the fluid impermeable barrier 102.

The two arms 140 may include a size that results in the technical effect of the two arms remaining selectively adhered to the pelvic region 152 of the user 150. As shown in FIG. 1C, the two arms 140 may be aligned laterally on the fluid impermeable barrier 102. Moreover, in some embodiments, the two arms may be coextensive, connected, or integral with one another. The adhesive surface 142 may extend continuously or uninterrupted across the two arms 140. Alternatively, the adhesive surface 142 of each arm 140 may cover only a portion of each arm 140 such that a gap between adhesive surface 142 of the two arms 140 is present. The adhesive surface 142 itself may include any of the dimensions described below in relation to the two arms 142. For example, the adhesive surface 142 may extend from the fluid impermeable barrier 102 any of the dimensions described below in relation to the extension of each of the two arms 140 from the fluid impermeable harrier 142. Furthermore, the adhesive surface 142 may include any width described below in relation to the maximum width W of each of the two arms 140.

Each of the two arms 140 may extend a predetermined distance from the fluid impermeable harrier 102. For example, each arm 140 may extend at least about 2.5 cm from the fluid impermeable barrier 102, at least about 3.8 cm from the fluid impermeable barrier 102, at least about 5.1 cm from the fluid impermeable barrier 102, at least about 6.4 cm from the fluid impermeable barrier 102, at least about 7.6 cm from the fluid impermeable barrier 102, at least about 8.9 cm from the fluid impermeable barrier 102, at least about 10.2 cm from the fluid impermeable barrier 102, at least about 11.4 cm from the fluid impermeable harrier 102, at least about 12.7 cm from the fluid impermeable harrier 102, at least about 15.2 cm from the fluid impermeable barrier 102, less than about 2.5 cm from the fluid impermeable barrier 102, less than about 5.1 cm from the fluid impermeable barrier 102, less than about 7.6 cm from the fluid impermeable barrier 102, less than about 10.2 cm from the fluid impermeable barrier 102, less than about 12.7 cm from the fluid impermeable barrier 102, less than about 15.2 cm from the fluid impermeable barrier 102, about 0.64 cm to about 2.5 cm from the fluid impermeable barrier 102, about 2.5 cm to about 5.1 cm from the fluid impermeable barrier 102, about 5.1 cm to about 7.6 cm from the fluid impermeable barrier 102, about 7.6 cm to about 10.2 cm from the fluid impermeable barrier 102, about 10.2 cm to about 12.7 cm from the fluid impermeable barrier 102, about 12.7 cm to about 15.2 cm from the fluid impermeable barrier 102, about 0.64 cm to about 5.1 cm from the fluid impermeable barrier 102, about 5.1 cm to about 10.2 cm from the fluid impermeable barrier 102, or about 10.2 cm to about 15.2 cm from the fluid impermeable barrier 102.

Each of the arms 140 also may include a width W that is measured on a line or plane parallel to the conduit 108 and/or a longitudinal axis of the fluid impermeable harrier 102. The width W may taper or decrease on each arm 140 as the arm 140 approaches the fluid impermeable barrier 102. Each of the arms 140 may include a maximum width W of at least about 1.3 cm, at least about 2.5 cm, at least about 3.8 cm, at least about 5.1 cm, at least about 6.4 cm, at least about 7.6 cm, at least about 8.9 cm, at least about 10.2 cm, less than about 15.2 cm, less than about 12.7 cm, less than about 10.2 cm, less than about 7.6 cm, less than about 5.1 cm, less than about 2.5 cm, less than about 1.3 cm, about 1.3 cm to about 2.5 cm, about 2.5 cm to about 5.1 cm, about 5.1 cm to about 7.6 cm, about 7.6 cm to about 10.2 cm, about 10.2 cm to about 12.7 cm, or about 12.7 cm to about 15.2 cm.

The arms 140 may be secured to the fluid impermeable barrier 102 with any suitable securement, such as an industrial grade adhesive, an ultrasonic bonding or welding, molded to the fluid impermeable barrier 102, a mechanical attachment, a strap, hook and loop fasteners, buttons, snaps, clips, magnets, a ring, or any combination thereof.

FIG. 1D is a cross-sectional view of the fluid collection device 100 taken along line 1-1 of FIG. 1A. The fluid collection device 100 may include a fluid permeable body 120 or layer disposed in the chamber 104. The fluid permeable body 120 may cover or extend across at least a portion (e.g., all) of the opening 106. The fluid permeable body 120 may be configured to wick any fluid away from the opening 106, thereby preventing the fluid from escaping the chamber 104. The fluid permeable body 120 also may wick the fluid generally towards an interior of the chamber 104, as discussed in more detail below. A portion of the fluid permeable body 120 may define a portion of an outer surface of the fluid collection device 100. Specifically, the portion of the fluid permeable body 120 defining the portion of the outer surface of the fluid collection device 100 may be the portion of the fluid permeable body 120 exposed by the opening 106 defined by the fluid impermeable barrier 102 that contacts the user. Moreover, the portion of the fluid permeable device defining the portion of the outer surface of the fluid collection device 100 may be free from coverage by gauze or other wicking material at the opening.

The fluid permeable body 120 may include any material that may wick the fluid. The permeable properties referred to herein may be wicking, capillary action, diffusion, or other similar properties or processes, and are referred to herein as “permeable” and/or “wicking.” Such “wicking” may exclude absorption into the wicking material. The fluid permeable body 120 may include a one-way fluid movement fabric. As such, the fluid permeable body 120 may remove fluid from the area around the female urethra, thereby leaving the urethra dry. The fluid permeable body 120 may enable the fluid to flow generally towards a reservoir 122 of void space formed within the chamber 104. For example, the fluid permeable body 120 may include a porous or fibrous material, such as hydrophilic polyolefin. In some embodiments, the fluid permeable body 120 consists of or consists essentially of a porous or fibrous material, such as hydrophilic polyolefin. Examples of polyolefin that may be used in the fluid permeable body 120 include, bmt are not limited to, polyethylene, polypropylene, polyisobutylene, ethylene propylene rubber, ethylene propylene diene monomer, or combinations thereof. The porous or fibrous material may be extruded into a substantially cylindrically shape to fit within the chamber 104 of the fluid impermeable barrier 102. The fluid permeable body 120 may include varying densities or dimensions. Moreover, the fluid permeable body 120 may be manufactured according to various manufacturing methods, such as molding, extrusion, or sintering.

In some embodiments, the fluid permeable body 120 includes a singular and porous body. That is, during use, the fluid permeable body 120 extends from the conduit 108 to interface the fluid impermeable barrier 102 and the opening 106. In some embodiments, a majority of the outer surface 109 (shown in FIG. 1E) of the fluid permeable body 120 interfaces with an inner surface 103 (shown in FIG. 1E) of the fluid impermeable barrier 106. A singular fluid permeable body 120 may be advantageous to conventional systems, which typically require an air-laid nonwoven pad covered by a ribbed fabric compression bandage, because a singular fluid permeable body 120 reduced the number of components in the fluid collection device 100, reduces the assembly time of the fluid collection device 100, requires shelf-life data for only a single component, and provides a latex-free single component. In some embodiments, at least a portion of the singular porous material of the fluid permeable body 120 extends continuously between the opening 106 and the reservoir 122 to wick any fluid from the opening 106 directly to the reservoir 122. Moreover, as the fluid impermeable barrier 102 is flexible and the fluid permeable body 120 is configured to wick fluid from the body rather than absorb fluid from the body and hold the fluid against the body, the fluid collection device 100, in some embodiments, is free from a seal or cushioning ring on the inward edge 129 defining the opening 106. In these and other embodiments, the fluid permeable body 120 includes an outer surface and a single layer or type of material between the opening 106 and the conduit 108 positioned within the fluid permeable body 120.

In other embodiments, the fluid permeable body 120 may include two or more layers of fluid permeable materials and include no (or an absence of) more than two layers of material between the opening 106 and the conduit 108 positioned within the fluid permeable body 120. For example, the fluid collection device 100 may include a fluid permeable membrane covering or wrapping around at least a portion of a fluid permeable body, with both the fluid permeable membrane and the fluid permeable body being disposed in the chamber 104. The fluid permeable membrane may cover or extend across at least a portion (e.g., all) of the opening 106. The fluid permeable membrane may be configured to wick any fluid away from the opening 106, thereby preventing the fluid from escaping the chamber 104. The permeable properties referred to herein may be wicking, capillary action, diffusion, or other similar properties or processes, and are referred to herein as “permeable” and/or “wicking.” In some embodiments, at least one of the fluid permeable membrane or the fluid permeable support include nylon configured to wick fluid away from the opening 106. The material of the fluid permeable membrane and the fluid permeable support also may include natural fibers. In such examples, the material may have a coating to prevent or limit absorption of fluid into the material, such as a water repellent coating. Such “wicking” may not include absorption into the wicking material. Put another way, substantially no absorption of fluid into the material may take place after the material is exposed to the fluid and removed from the fluid for a time. While no absorption is desired, the term “substantially no absorption” may allow for nominal amounts of absorption of fluid into the wicking material (e.g., absorbency), such as about 30 wt % of the dry weight of the wicking material, about 20 wt %, about 10 wt %, about 7 wt %, about 5 wt %, about 3 wt %, about 2 wt %, about 1 wt %, or about 0.5 wt % of the dry weight of the wicking material.

The fluid permeable membrane may also wick the fluid generally towards an interior of the chamber 104, as discussed in more detail below. The fluid permeable membrane may include any material that may wick the fluid. For example, the fluid permeable membrane may include fabric, such as a gauze (e.g., a silk, linen, polymer based materials such as polyester, or cotton gauze), another soft fabric (e.g., jersey knit fabric or the like), or another smooth fabric (e.g., rayon, satin, or the like). Forming the fluid permeable membrane from gauze, soft fabric, and/or smooth fabric may reduce chaffing caused by the fluid collection device 100. Other embodiments of fluid permeable membranes, fluid permeable supports, chambers, and their shapes and configurations are disclosed in U.S. patent application Ser. No. 15/612,325 filed on Jun. 2, 2017; U.S. patent application Ser. No. 15/260,103 filed on Sep. 8, 2016; U.S. patent application Ser. No. 15/611,587 filed on Jun. 1, 2017; PCT Patent Application No. PCT/US19/29608, filed on Apr. 29, 2019, the disclosure of each of which is incorporated herein, in its entirety, by this reference. In many embodiments, the fluid permeable body 120 includes a fluid permeable support including a porous nylon structure (e.g., spun nylon fibers) and a fluid permeable membrane including gauze about or over the porous nylon structure.

The fluid collection device 100 also includes conduit 108 that is at least partially disposed in the chamber 104. The conduit 108 (e.g., a tube) includes an inlet 110 at a second end region 127 of the fluid impermeable barrier 102 and an outlet 112 at a first end region 125 of the fluid impermeable barrier 102 positioned downstream from the inlet 110. The conduit 108 provides fluid communication between an interior region of the chamber 104 and a fluid storage container (not shown) or a portable vacuum source (not shown). For example, the conduit 108 may directly or indirectly fluidly couple the interior region of the chamber 104 and/or the reservoir 122 with the fluid storage container or the portable vacuum source.

In the illustrated embodiment, the fluid permeable body 120 defines a bore 202 extending through the fluid permeable body 120 from a first body end 121 of the fluid permeable body 120 to a second body end 123 of the fluid permeable body 120 distal to the first body end 120. In other embodiments, the bore 202 extends only partially into the fluid permeable body from the first body end 121 of the fluid permeable body 120.

In the illustrated embodiment, the conduit 108 is at least partially disposed in the chamber 104 and interfaces at least a portion of the bore 202 of the fluid permeable body 120. For example, the conduit 108 may extend into the fluid impermeable barrier 102 from the first end region 125 (e.g., proximate to the outlet 112) and may extend through the bore 202 to the second end region 127 (e.g., opposite the first end region 125) to a point proximate to the reservoir 122 such that the inlet 110 is in fluid communication with the reservoir 122. For example, in the illustrated embodiment, the inlet 110 is positioned in the reservoir 122. However, in other embodiments, the inlet 110 may be positioned flush with or behind an end of the fluid permeable body 120 that partially defines the reservoir 122. The fluid collected in the fluid collection device 100 may be removed from the interior region of the chamber 104 via the conduit 108. The conduit 108 may include a flexible material such as plastic tubing (e.g., medical tubing). Such plastic tubing may include a thermoplastic elastomer, polyvinyl chloride, ethylene vinyl acetate, polytetrafluoroethylene, etc., tubing. In some embodiments, the conduit 108 may include silicone or latex.

The fluid impermeable barrier 102 may store fluids in the reservoir 122 therein. The reservoir 122 is an unoccupied portion of the chamber 104 and is void of other material. In some embodiments, the reservoir 122 is defined at least partially by the fluid permeable body 120 and the fluid impermeable barrier 102. For example, in an embodiment, the reservoir 122 may be located at the portion of the chamber 104 that is closest to the inlet 110 (e.g., the second end region). Accordingly, in the embodiment in FIG. 1D, the reservoir 122 is defined by the second body end 123 of the fluid permeable body 120 and the second end region 127 of the fluid impermeable barrier 122. However, the reservoir 122 may be located at different locations in the chamber 104. For example, the reservoir 122 may be located at the end of the chamber 104 that is closest to the outlet 112. In these and other embodiments, the conduit 108 may extend through the first end region 125 of the fluid impermeable barrier 102 and to the reservoir 122 without extending through the fluid permeable body 120. Accordingly, in these and other embodiments, the fluid permeable body 120 may be free from the bore. In another embodiment, the fluid collection device 100 may include multiple reservoirs, such as a first reservoir that is located at the portion of the chamber of the chamber 104 that is closest to the inlet 110 (e.g., second end region) and a second reservoir that is located at the portion of the of the chamber 104 that is closest to the outlet 112 (e.g., first end region). In another example, the fluid permeable body 120 is spaced from at least a portion of the conduit 108 and the reservoir 122 may be the space between the fluid permeable body 120 and the conduit 108. Other embodiments of reservoirs, fluid impermeable barriers, fluid permeable membranes, fluid permeable bodies, chambers, and their shapes and configurations are disclosed in U.S. patent application Ser. No. 15/612,325 filed on Jun. 2, 2017; U.S. patent application Ser. No. 15/260,103 filed on Sep. 8, 2016; and U.S. patent application Ser. No. 15/611,587 filed on Jun. 1, 2017, the disclosure of each of which is incorporated herein, in its entirety, by this reference.

The fluid impermeable barrier 102 and the fluid permeable body 120 may be configured to have the conduit 108 at least partially disposed in the chamber 104. For example, the fluid permeable body 120 may be configured to form a space that accommodates the conduit 108, such as the bore 202. In another example, the fluid impermeable barrier 102 may define an aperture 124 sized to receive the conduit 108 (e.g., at least one tube). The at least one conduit 108 may be disposed in the chamber 104 via the aperture 124. The aperture 124 may be configured to form an at least substantially fluid tight seal against the conduit 108 or the at least one tube thereby substantially preventing the fluids from escaping the chamber 104.

In some embodiments, the conduit 108 may extend through the fluid permeable body 120 and at least partially into the reservoir 122, as shown in FIG. 1D. In some embodiments, the conduit 108 may extend through the fluid permeable body 120 and terminate at or before the second body end 123 of the fluid permeable body 120 such that the conduit 108 does not extend into the reservoir 122 (or the reservoir 122 is absent of the conduit 108). In other embodiments, the end of the conduit 108 may be recessed from the second body end 123 of the fluid permeable body 120. The end of the conduit 108 also may be selectively moveable between partially extending into the reservoir 122 and recessed from or flush with the second body end 123 of the fluid permeable body.

When secured to the fluid collection device 100, the conduit 108 is configured to provide fluid communication with and at least partially extend between one or more of a fluid storage containers (not shown) and a portable vacuum source (not shown). For example, the conduit 108 may be configured to be fluidly coupled to and at least partially extend between one or more of the fluid storage containers and the portable vacuum source. In an embodiment, the conduit 108 is configured to be directly connected to the portable vacuum source (not shown). In such an example, the conduit 108 may extend from the fluid impermeable barrier 102 by at least one foot, at least two feet, at least three feet, or at least six feet. In another example, the conduit 108 is configured to be indirectly connected to at least one of the fluid storage container (not shown) or the portable vacuum source (not shown). In some examples, the conduit may be frosted or opaque (e.g., black) to obscure visibility of the fluids therein. In some embodiments, the conduit is secured to a wearer's skin with a catheter securement device, such as a STATLOCK® catheter securement device available from C. R. Bard, Inc., including but not limited to those disclosed in U.S. Pat. Nos. 6,117,163; 6,123,398; and 8,211,063, the disclosures of which are all incorporated herein by reference in their entirety.

The inlet 110 and the outlet 112 are configured to provide fluid communication (e.g., directly or indirectly) between the portable vacuum source (not shown) and the chamber 104 (e.g., the reservoir 122). For example, the inlet 110 and the outlet 112 of the conduit 108 may be configured to directly or indirectly fluidly couple the portable vacuum source to the reservoir 122. In an embodiment, the inlet 110 and/or the outlet 112 may form a male connector. In another example, the inlet 110 and/or the outlet 112 may form a female connector. In an embodiment, the inlet 110 and/or the outlet 112 may include ribs that are configured to facilitate secure couplings. In an embodiment, the inlet 110 and/or the outlet 112 may form a tapered shape. In an embodiment, the inlet 110 and/or the outlet 112 may include a rigid or flexible material.

Locating the inlet 110 at or near a gravimetrically low point of the chamber 104 enables the conduit to receive more of the fluids than if inlet 110 was located elsewhere and reduce the likelihood of pooling (e.g., pooling of the fluids may cause microbe growth and foul odors). For instance, the fluids in the fluid permeable body 120 may flow in any direction due to capillary forces. However, the fluids may exhibit a preference to flow in the direction of gravity, especially when at least a portion of the fluid permeable body 120 is saturated with the fluids.

As the portable vacuum source applies a vacuum/suction in the conduit 108, the fluid(s) in the chamber 104 (e.g., such as in the reservoir 122 positioned at the first end region 125, the second end region 127, or other intermediary positions within the chamber 104) may be drawn into the inlet 110 and out of the fluid collection device 100 via the conduit 108.

In an embodiment, the conduit 108 is configured to be at least insertable into the chamber 104. In such an embodiment, the conduit 108 may include one or more markers 131 (shown in FIG. 1A) on an exterior thereof that are configured to facilitate insertion of the conduit 108 into the chamber 104. For example, the conduit 108 may include one or more markings thereon that are configured to prevent over or under insertion of the conduit 108, such as when the conduit 108 defines an inlet 110 that is configured to be disposed in or adjacent to the reservoir 122. In another embodiment, the conduit 108 may include one or more markings thereon that are configured to facilitate correct rotation of the conduit 108 relative to the chamber 104. In an embodiment, the one or more markings may include a line, a dot, a sticker, or any other suitable marking. In examples, the conduit 108 may extend into the fluid impermeable harrier 102 from the first end region (e.g., proximate to the outlet 112) and may extend to the second end region (e.g., opposite the first end region) to a point proximate to the reservoir 122 such that the inlet 110 is in fluid communication with the reservoir 122. In some embodiments (not shown), the conduit 108 may enter the second end region and the inlet 110 may be disposed in the second end region (e.g., in the reservoir 122). The fluid collected in the fluid collection device 100 may be removed from the interior region of the chamber 104 via the conduit 108. The conduit 108 may include a flexible material such as plastic tubing (e.g., medical tubing) as disclosed herein. In some examples, the conduit 108 may include one or more portions that atm resilient, such as having one or more of a diameter or wall thickness that allows the conduit to be flexible.

In an embodiment, one or more components of the fluid collection device 100 may include an antimicrobial material, such as an antibacterial material where the fluid collection device may contact the wearer or the bodily fluid of the wearer. The antimicrobial material may include an antimicrobial coating, such as a nitrofurazone or silver coating. The antimicrobial material may inhibit microbial growth, such as microbial growth due to pooling or stagnation of the fluids. In an embodiment, one or more components of the fluid collection device 100 (e.g., impermeable barrier 102, conduit 108, etc.) may include an odor blocking or absorbing material such as a cyclodextrine containing material or a thermoplastic elastomer (TPE) polymer.

In any of the embodiments disclosed herein, the conduits 108 may include or be operably coupled to a flow meter (not shown) to measure the flow of fluids therein, one or more securement devices (e.g., a StatLock securement device, not shown) or fittings to secure the conduit 108 to one or more components of the systems or devices disclosed herein (e.g., portable vacuum source or fluid storage container), or one or more valves to control the flow of fluids in the systems and devices herein. In an embodiment, at least one of portion of the conduit 108 of the fluid collection devices or systems herein may be formed of an at least partially opaque material which may obscure the fluids that are present therein. For example, a first section of the conduit 108 disclosed herein may be formed of an opaque material or translucent material while a second section of the conduit 108 may be formed of a transparent material or translucent material. In some embodiments, the first section may include transparent or translucent material. Unlike the opaque or nearly opaque material, the translucent material allows a user of the devices and systems herein to visually identify fluids or issues that are inhibiting the flow of fluids within the conduit 108.

In any of the examples, systems or devices disclosed herein, the system of fluid collection device may include moisture sensors (not shown) disposed inside of the chamber of the fluid collection device. In such examples, the moisture sensor may be operably coupled to a controller or directly to the portable vacuum source, and may provide electrical signals indicating that moisture is or is not detected in one or more portions of the chamber. The moisture sensor(s) may provide an indication that moisture is present, and responsive thereto, the controller or portable vacuum device may direct the initiation of suction to the chamber to remove the fluid therefrom. Suitable moisture sensors may include capacitance sensors, volumetric sensors, potential sensors, resistance sensors, frequency domain reflectometry sensors, time domain reflectometry sensors, or any other suitable moisture sensor. In practice, the moisture sensors may detect moisture in the chamber and may provide a signal to the controller or portable vacuum source to activate the portable suction device.

FIG. 1F is an isometric view of a fluid collection device 100a having a fluid impermeable barrier 102, a fluid permeable body 120, a conduit 108, and two arms 140a having an adhesive surface 142a. Unless otherwise noted, the fluid collection device 100a may include any of the features, elements, compositions, positioning, dimensions, etc. described above in relation to the fluid collection device 100, including the fluid impermeable barrier 102, the fluid permeable body 120, the conduit 108, the reservoir 122, etc. Moreover, the two arms 140a and the adhesive surface 142a on each of the two arms 140a may include any of the features, elements, compositions, positioning, dimensions described above in relation to the two arms 140 and the adhesive surface 142 on each of the two arms 140.

In addition to the two arms 140a each having the adhesive surface 142a, the adhesive securement feature of the fluid collection device 100a may include a ring 148 or strap configured to slide onto and/or fit around at least a portion of the fluid impermeable barrier 102. For example, in FIG. 1F, the ring 148 is positioned around fluid impermeable barrier 102 and the first end region 125 of the fluid impermeable barrier 102 between the opening 106 and the aperture 124. In use, with the ring 148 positioned around at least a portion of the first end region 125 of the fluid impermeable barrier, the fluid collection device 100a may be positioned on the user 150 similar to that shown in FIG. 1B. In other embodiments, the ring 148 may be positioned around other regions of the fluid impermeable barrier 102, including the second end region 127 between the opening 106 and the distal end of the fluid impermeable barrier 102.

The ring 148 results in the technical effect of one or more adhesive securement features (e.g., the two arms 140a each having the adhesive surface 142a) that are detachably securable to the fluid impermeable barrier 102 of the fluid collection device 100a. Accordingly, the ring 148 and the two arms 140a may be secured to the fluid impermeable harrier 102 only when determined to be necessary or helpful by the user 150 or a caregiver. The ring 148 also results in the technical effect of allowing the user 150 or the caregiver to position the two arms 140a in a selected, preferred location on the fluid impermeable barrier that is most likely to fit the user 150. In many embodiments, the arms 140a are attached to the ting 148, and the ring 148 is initially separate from the fluid impermeable barrier 102, allowing the user 150 or a caregiver to slide or other move the ring 148 and the arms 140a onto a preferred or user-determined region of the fluid impermeable barrier 102, such as the first end region 125 or the second end region 127. The ring 148 may be sized to slide or move the arms 140a to a preferred region on the fluid impermeable barrier 102, and then remain in place on the fluid impermeable barrier 102 to secure the fluid collection device 100a to the user 150 with the adhesive surfaces 142a of the arms 140a. Accordingly, the ring 148 may have an inner diameter or circumference that is substantially equal to an outer diameter or circumference of the fluid impermeable barrier 102. The ring 148 may include a material similar or equal to the fluid impermeable barrier 102, such as such as a fluid impermeable polymer (e.g., silicone, polypropylene, polyethylene, polyethylene terephthalate, a polycarbonate, etc.), polyurethane films, thermoplastic elastomer (TPE), rubber, thermoplastic polyurethane, another suitable material, or combinations thereof. In some embodiments, the ring 148 may include a generally elastic material such that the ring 148 stretches to fit around the fluid impermeable barrier 102 and remains in place on the fluid impermeable harrier 102 once positioned by the user 150 or a caregiver. The arms 140a may be attached to the ring 148 with any suitable securement, such as an industrial grade adhesive, an ultrasonic bonding or welding, molded to the ring 148, a mechanical attachment, a strap, hook and loop fasteners, buttons, snaps, clips, magnets, a ring, or any combination thereof. The ring 148 also may lower costs and improve efficiency of manufacture of the urine collection device 100.

FIG. 1G is a front view of the female user 150 with a fluid collection device 100b secured to the female user 150. Unless otherwise noted, the fluid collection device 100b may include any of the features, elements, compositions, positioning, dimensions, etc. described above in relation to the fluid collection device 100, including the fluid impermeable barrier 102, the fluid permeable body 120, the conduit 108, the reservoir 122, etc. Moreover, the two arms 140a and the adhesive surface 142a on each of the two arms 140a may include any of the features, elements, compositions, positioning, and/or dimensions described above in relation to the two arms 140 and the adhesive surface 142 on each of the two arms 140.

As noted above, the ring 148 may be positioned around other regions of the fluid impermeable barrier 102, including the second end region 127 between the opening 106 and the distal end of the fluid impermeable barrier 102. FIG. 1G shows a fluid collection device 100b positioned on the user 150 when the ring 148 is positioned around the second end region 127 of the fluid impermeable barrier 102. When the ring 148 is positioned around the second end region 127 of the fluid impermeable barrier 102, the adhesive surfaces 142a of the arms 140a may attach to the inner thighs 156 of the user 150. Moreover. FIG. 1G shows exemplary attachment of the fluid collection device 100 if the arms 140 are positioned on the fluid permeable barrier 102 proximate to the second end region 127.

In some embodiments, an adhesive securement feature may be positioned on the conduit of a fluid collection device in addition or alternative to positioning an adhesive securement feature on the fluid impermeable barrier of the fluid collection device. Positioning the adhesive securement feature on the conduit may provide the technical effect of securing the fluid collection device to user at the lower abdominal region of the user where less hair may be present and/or the adhesive securement feature is more likely to remain adhered to the user. Positioning the adhesive securement feature on the conduit also may lower costs and improve efficiency of manufacture of the urine collection device.

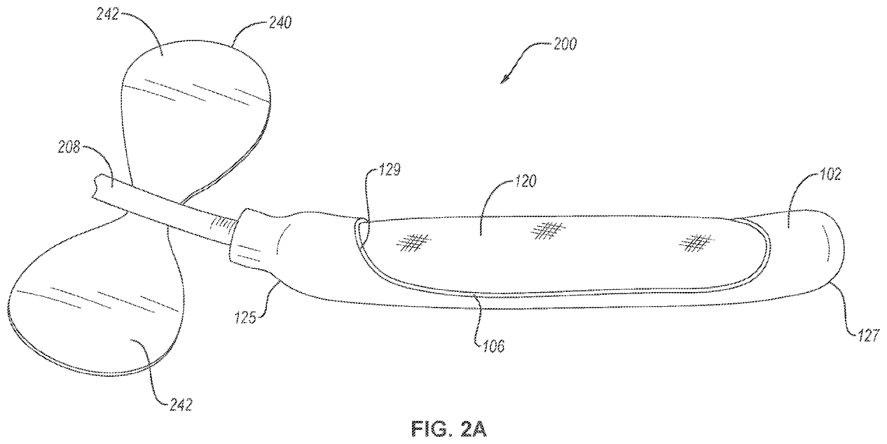

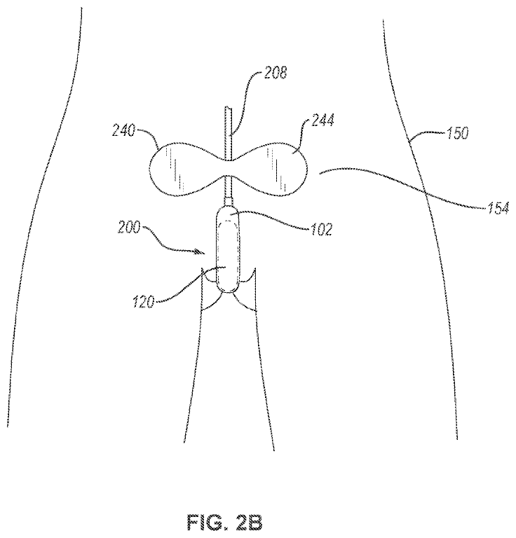

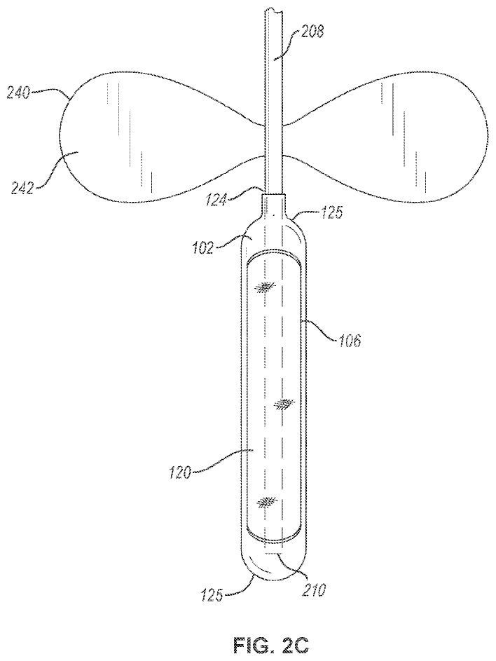

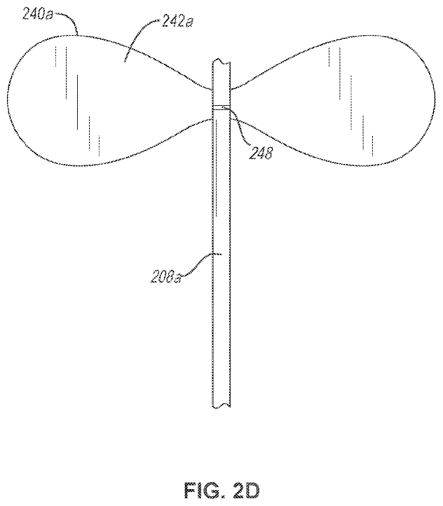

FIG. 2A is an isometric view of a fluid collection device 200 having a fluid impermeable harrier 102, a fluid permeable body 120, a conduit 208, and two arms 240 positioned on the conduit 208, each of the arms 240 having an adhesive surface 242. Unless otherwise noted, the fluid collection device 200 may include any of the features, elements, compositions, positioning, dimensions, etc. described above in relation to the fluid collection devices 100, 100a, or 100b, including the fluid impermeable barrier 102, the fluid permeable body 120, the reservoir 122, etc. Unless otherwise noted, the conduit 208 may include any of the features, elements, compositions, positioning, dimensions, etc., of the conduit 108 described above. Moreover, the two arms 240 and the adhesive surface 242 on each of the two arms 240 may include any of the features, elements, compositions, positioning, and/or dimensions descried above in relation to the two arms 140 and the adhesive surface 142 on each of the two arms 140. Moreover, though not shown in FIG. 2A, in addition to the arms 240, the fluid collection device 200 also may include one or more arms 140 positioned on the fluid impermeable barrier 102 as described above in relation to the fluid collection devices 100, 100a, and 100b.

In an embodiment, fluid collection device 200 includes an adhesive securement feature secured or securable to the conduit 208 and configured to attach the fluid collection device 200 to an individual. For example, the fluid collection device 200 shown in FIG. 2A includes one or more (e.g., two) arms 240 or wings configured to attach or secure the fluid collection device 200 to an individual. The arms 240 may each include an adhesive surface 242 oriented generally in the same direction as the opening 106 is oriented. The adhesive surface 242 may include an adhesive material, such as a glue, contact adhesive, epoxy, hydrogel adhesive, tape, an acrylic adhesive, a silicone adhesive, a hydrogel adhesive, other adhesives suitable for placement on the body of a user or fahric worn by the user, or any combination thereof. For example, the adhesive may include an acrylate (e.g., methacrylate or epoxy diacrylate) or any other adhesive suitable for use on bandages. The arms 240 also may include a removable cover secured to the adhesive layer 242 that may be removed prior to securing the adhesive layer to the user.

FIG. 2B is a front view of the female user 150 with the fluid collection device 200 secured to the female user 150. In use, the fluid permeable body 120 of the fluid collection device is positioned adjacent to a urethra of the user 150. The fluid permeable body 120 is disposed within a chamber 104 (shown in FIG. 1D) of the fluid impermeable barrier 102 of the fluid collection device 100 and is exposed to the urethra of the user 150 through the opening 106 in the fluid collection device 100. Fluids received in the chamber 104 of the fluid collection device 100 from the urethra may be removed through the conduit 108.

The adhesive surface 242 of each of the arms 240 may attach the fluid collection device 200 to the user 150, and the arms 240 also may include a non-adhesive surface 244 opposite or distal to the adhesive surface 242. The adhesive surface 242 may be attached directly to the skin of the user 150 or may attach to the fabric worn by the user 150, such as undergarments. The arms 240 may be positioned at various areas on the conduit 208 to secure the fluid collection device 200 directly to a predetermined area of the body of the user, such as the abdominal region, the pubic region, the pelvic region, or the hypogastrium (or hypogastric) region. In FIG. 2B, the arms 240 are positioned on the conduit 208 effective allow the adhesive surface 242 of each of the arms 240 to attach to the lower abdominal region 154 of the user 150.

Turning to FIG. 2C, which shows a front view of a fluid collection device 200. In some embodiments, the two arms 240 are positioned on the conduit 208 on a rear or back surface of the conduit 208, generally distal to the opening 106. In this position, the conduit 208 is positioned between the arms 240 and the user 150 when the fluid collection device 200 is attached to the user 150. In other embodiments, the arms 240 are positioned on the conduit 208 on a front surface of the conduit 208 such that at least a portion of the arms 208 are positioned between the conduit 208 and the user 150 when the fluid collection device 200 is attached to the user 150.

The arms 240 may be positioned at various positions on the conduit 208 relative to the inlet 210 of the conduit 208 positioned within the chamber 104 of the fluid impermeable barrier 102, effective to allow the arms 240 to be attached to other areas of the body of the user 150. For example, in various embodiments, the arms 240 may be positioned on the conduit 208 to be less than about 30.5 cm from the inlet 210, less than about 27.9 cm from the inlet 210, less than about 25.4 cm from the inlet 210, less than about 22.9 cm from the inlet 210, less than about 20.3 cm from inlet 210, less than about 17.8 cm from inlet 210, less than about 15.2 cm from the inlet 210, more than about 15.2 cm from the inlet 210, more than about 17.8 cm from the inlet 210, more than about 20.3 cm from the inlet 210, more than about 22.9 cm from the inlet 210, more than about 25.4 cm from the inlet 210, more than about 27.9 cm from the inlet 210, more than about 30.5 cm from the inlet 210, more than about 33.0 cm from the inlet 210, about 15.2 cm to about 17.8 cm from the inlet 210, about 17.8 cm to about 20.3 cm from the inlet 210, about 20.3 cm to about 22.9 cm from the inlet 210, about 22.9 cm to about 25.4 cm from the inlet 210, about 25.4 cm to about 27.9 cm from inlet 210, about 27.9 cm to about 30.5 cm from the inlet 210, about 30.5 cm to about 33.0 cm from the inlet 210, about 33.0 cm to about 35.6 cm from the inlet, about 35.6 cm to about 38.1 cm from the inlet 210, about 15.2 cm to about 22.9 cm from the inlet 210, about 22.9 cm to about 30.5 cm from the inlet 210, about 30.5 cm to about 38.1 cm from the inlet 210, about 15.2 cm to about 30.5 cm from the inlet, about 15.2 cm from the inlet 210, about 17.8 cm from the inlet 210, about 20.3 cm from the inlet 210, about 22.9 cm from the inlet 210, about 25.4 cm from the inlet 210, about 27.9 cm from the inlet 210, about 30.5 cm from the inlet 210, about 33.0 cm from the inlet 210, about 35.6 cm from the inlet 210, or about 38.1 cm from the inlet 210. In many embodiments, the conduit 208 is slidable within the aperture 124 and the chamber 104 of the fluid impermeable barrier 102, effective to allow the arms 240 to be adjusted relative to the inlet 210.

As shown in FIG. 2C, the two arms 240 may be aligned laterally on the conduit 208. Moreover, in some embodiments, the two arms 240 may be coextensive, connected, or integral with one another. The adhesive surface 242 may extend continuously or uninterrupted across the two arms 240. Alternatively, the adhesive surface 242 of each arm 240 may cover only a portion of each arm 240 such that a gap between adhesive surface 242 of the two arms 240 is present. Each of the two arms 240 may extend a predetermined distance from the conduit 208. For example, each arm 240 may extend at least about 2.5 cm from the conduit 208, at least about 3.8 cm from the conduit 208, at least about 5.1 cm from the conduit 208, at least about 6.4 cm from the conduit 208, at least about 7.6 cm from the conduit 208, at least about 8.9 cm from the conduit 208, at least about 10.2 cm from the conduit 208, at least about 11.4 cm from the conduit 208, at least about 12.7 cm from the conduit 208, at least about 15.2 cm from the conduit 208, less than about 2.5 cm from the conduit 208, less than about 5.1 cm from the conduit 208, less than about 7.6 cm from the conduit 208, less than about 10.2 cm from the conduit 208, less than about 12.7 cm from the conduit 208, less than about 15.2 cm from the conduit 208, about 0.64 cm to about 2.5 cm from the conduit 208, about 2.5 cm to about 5.1 cm from the conduit 208, about 5.1 cm to about 7.6 cm from the conduit 208, about 7.6 cm to about 10.2 cm from the conduit 208, about 10.2 cm to about 12.7 cm from the conduit 208, about 12.7 cm to about 15.2 cm from the conduit 208, about 0.64 cm to about 5.1 cm from the conduit 208, about 5.1 cm to about 10.2 cm from the conduit 208, or about 10.2 cm to about 15.2 cm from the conduit 208. Each of the arms 240 also may include a width that is measured on a line or plane parallel to an axis of the fluid impermeable barrier 102. The width W may taper or decrease on each arm 240 as the arm 240 approaches the conduit 208. Each of the arms 240 may include a maximum width of any of the widths W described above in relation to the arms 140.

The arms 240 may be secured to the conduit 208 with any suitable securement, such as an industrial grade adhesive, an ultrasonic bonding or welding, molded to the conduit 208, a mechanical attachment, a strap, hook and loop fasteners, buttons, snaps, clips, magnets, a ring, or any combination thereof. Turning to FIG. 2D, in some embodiments the arms 240 may be secured to the conduit 208 with a ring 248 or a strap. The ring 248 or strap may be configured to slide onto and fit around at least a portion of the conduit 208. In many embodiments, the ring 248 is initially separate from the conduit 208, allowing the user 150 or a caregiver to slide the ring 248 onto a preferred or user-determined region of the conduit 208. The ring 248 may be sized to result in the technical effect of allowing a user or caregiver slide to a preferred region on the conduit 208, and then remain in place on conduit 208 to secure the fluid collection device 100 to the user 150 with the adhesive surfaces 242a of the arms 240a. Accordingly, the ring 248 may have an inner diameter or circumference that is substantially equal to an outer diameter or circumference of the conduit 208. The ring 248 may include a material similar or equal to the conduit 208. In some embodiments, the ring 248 may include a generally elastic material such that the ring 248 results in the technical effect of stretching to fit around the conduit 208 and remains in place on the conduit 208 once positioned by the user 150 or a caregiver. The ring 248 also may result in the technical effect of reducing costs and improving efficiency of manufacture of the urine collection device 200.

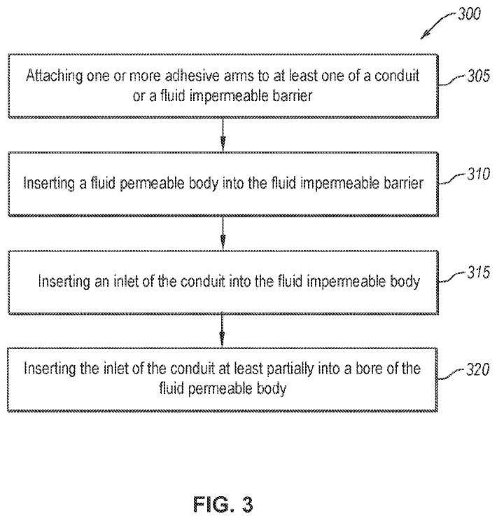

FIG. 3 is a flow diagram of a method 300 of assembling the fluid collection devices and/or fluid collection systems disclosed herein, according to an embodiment. The method 300 can include act 305, which recites “attaching one or more adhesive arms to at least one of a conduit or a fluid impermeable barrier” of a fluid collection device. Act 305 may be followed by act 310, which recites “inserting a fluid permeable body into the fluid impermeable barrier.” Act 310 may be followed by act 315, which recites “inserting an inlet of the conduit into the fluid impermeable body.” Act 315 may be followed by act 320, which recites “inserting the inlet of the conduit at least partially into a bore of the fluid permeable body.”

Acts 305, 310, 315, and 320 of the method 300 are for illustrative purposes. For example, the acts 305, 310, 315, and 320 of the method 300 can be performed in different orders, split into multiple acts, modified, supplemented, or combined. In an example, one or more of the acts 305, 310, 315, and 320 of the method 300 can be omitted from the method 300. For example, the method 300 may not include the act 305. Any of the acts 305, 310, 315, and 320 can include using any of the fluid collection devices, adhesive securement features, vacuum sources, fluid storage containers, systems, or components of the same disclosed herein. For example, the fluid collection device may include one or more arms having an adhesive surface. The fluid collection device may include a fluid impermeable barrier at least partially defining the chamber, the fluid impermeable barrier also defining an opening extending therethrough, the opening configured to be positioned adjacent to a female urethra or have a male urethra positioned therethrough. The fluid collection device may include a wicking material disposed with the chamber and a conduit disposed within wicking material, the conduit including an inlet positioned within the fluid collection device and an outlet.

Act 305 recites “attaching one or more adhesive arms to at least one of a conduit or a fluid impermeable barrier” of a fluid collection device. Attaching one or more adhesive arms to at least one of the conduit or the fluid impermeable barrier may include attaching two arms each having an adhesive surface to the at least one of the conduit or the fluid impermeable barrier of the fluid collection device. In some embodiments, attaching the two arms includes fitting a ring or a strap on the conduit, the two arms being secured to the ring. In some embodiments, attaching the two arms includes fitting a ring or a strap on the first end region or the second end region of the fluid impermeable barrier of the fluid collection device. The fluid impermeable barrier of the fluid collection device at least partially defines a chamber and also an opening extending therethrough. The opening is configured to be positioned adjacent to a female urethra or have a male urethra positioned therethrough. The fluid permeable body may include a singular porous hydrophilic polyolefin material extruded, molded, or sintered to a substantially cylindrical shape.

Act 310 recites “inserting a fluid permeable body into the fluid impermeable barrier.” Act 310 may include inserting a substantially cylindrical and fluid permeable body into the chamber of the fluid impermeable harrier. When the fluid permeable body is inserted into the chamber of the fluid impermeable barrier, the fluid permeable body interfaces at least a portion of the fluid impermeable barrier and covers at least a portion of the opening. The fluid permeable body includes a singular porous material that is substantially cylindrical in shape and configured to wick any fluid away from the opening. In some embodiments, act 310 may include inserting the fluid permeable body into the chamber of the fluid impermeable harrier such that a reservoir is defined within the chamber by a second body end of the fluid permeable body distal to the first body end and a second end region of the fluid impermeable barrier distal to the aperture. In some embodiments, act 310 may include inserting the substantially cylindrical and fluid permeable body into the chamber of the fluid impermeable barrier such that the fluid permeable body and the conduit fill substantially all of the chamber.

Act 315 recites “inserting an inlet of the conduit into the fluid impermeable body.” The conduit may be inserted into the fluid impermeable body through an aperture defined by the fluid impermeable barrier at a first end region of the fluid impermeable barrier. In some embodiments, act 315 may include inserting the inlet of the conduit into the bore at the first body end, through the bore of the fluid permeable body, through the second body end of the fluid permeable body, and into the reservoir such that the conduit extends from the reservoir, through the fluid permeable body, through the aperture to outside the fluid impermeable barrier.

Act 320 recites “inserting the inlet of the conduit at least partially into a bore of the fluid permeable body.” The bore extends at least partially through the fluid permeable body and is defined by the fluid permeable body. The conduit interfaces at least a portion of the fluid permeable body.

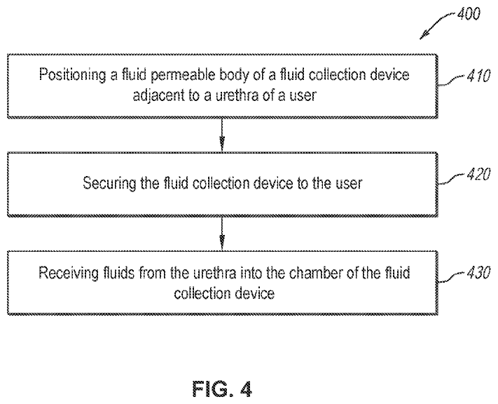

FIG. 4 is a flow diagram of a method 400 to collect fluid, according to an embodiment. The method 400 can include act 410, which recites “positioning a fluid permeable body of a fluid collection device adjacent to a urethra of a user.” Act 410 may be followed by act 420, which recites “securing the fluid collection device to the user.” Act 420 may be followed by act 430, which recites “receiving fluids from the urethra into a chamber of the fluid collection device.”

Acts 410, 420, 430 of the method 400 are for illustrative purposes. For example, the act 410, 420, 430 of the method 400 can be performed in different orders, split into multiple acts, modified, supplemented, or combined. In an example, one or more of the acts 410, 420, 430 of the method 400 can be omitted from the method 400. For example, the method 400 may not include the act 410. Any of the acts 410, 420, and 430 can include using any of the fluid collection devices, adhesive securement features, vacuum sources, fluid storage containers, systems, or components of the same disclosed herein. For example, the fluid collection device may include one or more arms having an adhesive surface. The fluid collection device may include a fluid impermeable barrier at least partially defining the chamber, the fluid impermeable barrier also defining an opening extending therethrough, the opening configured to be positioned adjacent to a female urethra or have a male urethra positioned therethrough. The fluid collection device may include a wicking material disposed with the chamber and a conduit disposed within wicking material, the conduit including an inlet positioned within the fluid collection device and an outlet.

Act 410 recites “positioning a fluid permeable body of a fluid collection device adjacent to a urethra of a user.” The fluid permeable body is disposed within a chamber of a fluid impermeable barrier of the fluid collection device and exposed to the female urethra of the user through an opening in the fluid collection device defined by the fluid impermeable barrier. The fluid collection device may be a female fluid collection device or a male fluid collection device.

Act 420 recites “securing the fluid collection device to the user.” Securing the fluid collection device to the user can include applying an adhesive surface of one or more arms of the fluid collection device to at least one of a pubic region, a pelvic region, or an abdominal region of the user effective to secure the fluid collection device to the user. The one or more adhesive arms may be secured to at least one of the fluid impermeable barrier or the conduit of the fluid collection device. In some embodiments, the method 400 also may include attaching two arms each having an adhesive surface to the at least one of the conduit or the fluid impermeable barrier of the fluid collection device before securing the fluid collection device to the user. Attaching the two arms may include fitting a ring or a strap on the conduit, the two arms being secured to the ring. Attaching the two arms may include fitting a ring or a strap on the first end region or the second end region of the fluid impermeable barrier of the fluid collection device.

Act 430 recites “receiving fluids from the urethra into a chamber of the fluid collection device.” For example, act 430 can include wicking the fluids away from urethra via the opening using wicking material (e.g., fluid permeable membrane and a fluid permeable support). In some examples, act 430 can include receiving the fluids into the chamber of the fluid collection device. In either example, act 430 can include flowing the fluid towards a portion of the chamber that is in fluid communication with an inlet of a conduit in fluid communication a vacuum source. For instance, act 430 can include flowing the fluids to a substantially unoccupied portion of the chamber (e.g., a reservoir), to a gravimetrically low point of the chamber, etc.

The method 400 may include applying suction with a vacuum source effective to suction the fluids from the chamber via a conduit disposed therein that is in fluid communication with the vacuum source. In examples, applying suction with a vacuum source effective to suction the fluids from the chamber via a conduit disposed therein that is in fluid communication with the vacuum source can include using any of the vacuum sources disclosed herein. In an example, applying suction can include activating the vacuum source (e.g., suction device) in fluid communication with the inlet of the conduit in the fluid collection device. In examples, activating the vacuum source in fluid communication with the inlet of the conduit in the fluid collection device can include supplying power to the vacuum source by one or more of flipping an on/off switch, pressing a button, plugging the vacuum source into a power outlet, putting batteries into the vacuum source, etc. In examples, the vacuum source may include a hand operated vacuum pump and applying suction with a vacuum source may include manually operating the hand operated vacuum pump effective to suction the fluids from the chamber via the conduit disposed therein that is in fluid communication with the vacuum source.

In examples, applying suction with a vacuum source effective to suction the fluids from the chamber via a conduit disposed therein that is in fluid communication with the vacuum source can be effective to remove at least some fluid (e.g., urine) from the chamber (e.g., interior region) of the fluid collection device. In examples, applying suction with a vacuum source effective to suction the fluids from the chamber via a conduit disposed therein that is in fluid communication with the vacuum source can be effective to transfer at least some of the fluid from the chamber of the fluid collection device to a fluid storage container (e.g., a bottle or hag) in fluid communication with the vacuum source and the fluid collection device. In examples, the vacuum source may be spaced from the fluid collection device.

In examples, applying suction with a vacuum source effective to suction the fluids from the chamber via a conduit disposed therein that is in fluid communication with the vacuum source may include detecting moisture in the chamber (e.g., via one or more moisture sensors) and responsive thereto, activating the vacuum source to provide suction in the chamber. Control of the vacuum source responsive to the signals indicating that moisture or a threshold level thereof is present in the chamber can be automatic, such as via a controller, or may merely provide an indication that a level of moisture is present that may necessitate removal of fluid from the chamber of the fluid collection device. In the latter case, a user may receive the indication and activate the vacuum pump.

In an example, the method 400 can include collecting the fluids that are removed from the fluid collection device, such as into a fluid storage container that is spaced from the fluid collection device that is in fluid communication with the conduit. The fluid storage container can include any of the fluid storage containers disclosed herein.

Devices and methods described herein can be configured to collect urine from a male user, such as having a fluid collection device shaped and sized to receive a male urethra (e.g., penis) therein. In examples, the method 400 can include positioning a receptacle of a male fluid collection device around the male urethra such that the male urethra is positioned in the receptacle.

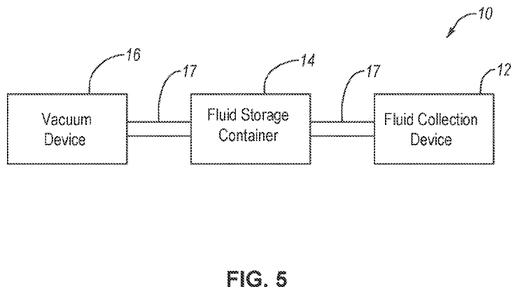

FIG. 5 is a block diagram of a system 10 for fluid collection, according to an embodiment. The system 10 includes a fluid collection device 12, a fluid storage container 14, and a vacuum source 16. The fluid collection device 12 may include any of the fluid collection devices described herein, such as the fluid collection device 100, 100a, 100b, or 200. The fluid collection device 12, the fluid storage container 14, and the vacuum source 16 may be fluidly coupled to each other via one or more conduits 17. The conduit 17 may include any of the conduits described herein, such as the conduit 108 or the conduit 208. The fluid collection device 12 may be operably coupled to one or more of the fluid storage container 14 or the portable vacuum source via the conduit 17. Fluid (e.g., urine or other bodily fluids) collected in the fluid collection device 12 may be removed from the fluid collection device 12 via the conduit 17, which protrudes into an interior region of the fluid collection device 12. For example, a first open end of the conduit 17 may extend into the fluid collection device 12 to a reservoir therein. The second open end of the conduit 17 may extend into the fluid storage container 14 or the vacuum source 16. The suction force may be introduced into the interior region of the fluid collection device 12 via the first open end of the conduit 17 responsive to a suction (e.g., vacuum) force applied at the second end of the conduit 17. The suction force may be applied to the second open end of the conduit 17 by the vacuum source 16 either directly or indirectly.

The suction force may be applied indirectly via the fluid storage container 14. For example, the second open end of the conduit 17 may be disposed within the fluid storage container 14 and an additional conduit 17 may extend from the fluid storage container 14 to the vacuum source 16. Accordingly, the vacuum source 16 may apply suction to the fluid collection device 12 via the fluid storage container 14. The suction force may be applied directly via the fluid storage container 14. For example, the second open end of the conduit 17 may be disposed within the vacuum source 16. An additional conduit 17 may extend from the vacuum source 16 to a point outside of the fluid collection device 12, such as to the fluid storage container 14. In such examples, the vacuum source 16 may be disposed between the fluid collection device 12 and the fluid storage container 14.

The fluid collection device 12 may be shaped and sized to be positioned adjacent to a female urethra. The fluid collection member of the fluid collection device 12 may include a fluid impermeable barrier at least partially defining a chamber (e.g., interior region of the fluid collection device member) of the fluid collection device 12. As described in more detail above, the fluid collection device 12 may include a softer, thinner fluid impermeable barrier than conventional fluid collection devices. The fluid impermeable barrier also defines an opening extending therethrough from the external environment. The opening may be positioned on the fluid collection member to be aligned adjacent to a female urethra. The fluid collection member of the fluid collection device 12 may include a fluid permeable body disposed within the fluid impermeable barrier. The fluid permeably body may include a fluid permeable membrane and fluid permeable support disposed within the fluid permeable membrane. The conduit 17 may extend into the fluid collection device 12 at a first end region, through one or more of the fluid impermeable barrier, fluid permeable membrane, or the fluid permeable support to a second end region of the fluid collection member of the fluid collection device 12. Example fluid collection devices for use with the systems and methods herein are described in more detail below.

In some embodiments, the fluid storage container 14 may include a bag (e.g., drainage bag), a bottle or cup (e.g., collection jar), or any other enclosed container for storing bodily fluids such as urine. In examples, the conduit 17 may extend from the fluid collection device 12 and attach to the fluid storage container 14 at a first point therein. An additional conduit 17 may attach to the fluid storage container 14 at a second point thereon and may extend and attach to the vacuum source 16. For example, the fluid storage container 14 may include a container fluidly coupled to a first conduit section that is also fluidly coupled to the fluid collection member of the fluid collection device 12. The container may be fluidly coupled to a second section of the conduit 17 that is also fluidly coupled to a portable vacuum source. In such examples, the portable vacuum source 16 may provide a vacuum/suction through the container to the fluid collection member to provide suction in the chamber of the fluid collection member. Accordingly, a vacuum (e.g., suction) may be drawn through fluid collection device 12 via the fluid storage container 14. As the fluid is drained from the chamber, the fluid may travel through the first section of conduit to the fluid storage container where it may be retained. Fluid, such as urine, may be drained from the fluid collection device 12 using the portable vacuum source 16.

In some embodiments, the vacuum source 16 may be disposed in or on the fluid collection device 12. In such examples, the conduit 17 may extend from the fluid collection device and attach to the vacuum source 16 at a first point therein. An additional conduit 17 may attach to the vacuum source 16 at a second point thereon and may extend out of the fluid collection device 12, and may attach to the fluid storage container 14. Accordingly, a vacuum (e.g., suction) may be drawn through fluid collection device 12 via the fluid storage container 14.

The vacuum source 16 may be portable and may include one or more of a manual vacuum pump, and electric vacuum pump, a diaphragm pump, a centrifugal pump, a displacement pump, a magnetically driven pump, a peristaltic pump, or any pump configured to produce a vacuum. The vacuum source 16 may provide a vacuum or suction to remove fluid from the fluid collection member of the fluid collection device 12. In some embodiments, the vacuum source 16 may be powered by one or more of a power cord (e.g., connected to a power socket), one or more batteries, or even manual power (e.g., a hand operated vacuum pump). In examples, the vacuum source 16 may be sized and shaped to fit outside of, on, or within the fluid collection device 12. For example, the vacuum source 16 may include one or more miniaturized pumps or one or more micro pumps. The portable vacuum sources 16 disclosed herein may include one or more of a switch, a button, a plug, a remote, or any other device suitable to activate the vacuum source 16. It should be understood that the vacuum sources 16 disclosed herein may provide a portable means of providing a suction or vacuum that allows use of the devices and systems herein outside of hospital or care facility environments where vacuum lines are plumbed into patient rooms or large (e.g., larger or heavier than a patient can readily carry) vacuum sources are located. For example, a portable vacuum source may be small and light enough to be carried by a user (e.g., patient) or aid (e.g., nurse) during transportation of the user.

As used herein, the term “about” or “substantially” refers to an allowable variance of the term modified by “about” by ±10% or +5%. Further, the terms “les % than,” “or less.” “greater than”, “more than,” or “or more” include as an endpoint, the value that is modified by the terms “less than,” “or less.” “greater than,” “more than.” or “or more.”

While various aspects and embodiments have been disclosed herein, other aspects and embodiments are contemplated. The various aspects and embodiment disclosed herein are for purposes of illustration and are not intended to be limiting.

1. A fluid collection device, comprising:
a fluid impermeable barrier at least partially defining a chamber, an opening extending longitudinally along the fluid impermeable barrier and configured to be positioned at least proximate to a urethra of a user, and an aperture configured to receive a conduit therethrough;
a fluid permeable body positioned at least partially within the chamber to extend across at least a portion of the opening and configured to wick fluid away from the opening; and
one or more arms including an adhesive surface oriented towards the fluid impermeable barrier, the one or more arms being one of (1) detachably secured or securable to the fluid impermeable barrier in a position selected by the user or a caregiver or (2) secured or securable to the conduit, the conduit being configured to be inserted into the aperture,
wherein the one or more arms are positioned to attach to at least one of an abdominal region, a pubic region, or a pelvic region of the user when the opening of the fluid impermeable barrier is positioned at least proximate to the urethra of the user.

2. The fluid collection device of claim 1, wherein the one or more arms include two arms aligned laterally.

3. The fluid collection device of claim 2, wherein the two arms are connected to one another.

4. The fluid collection device of claim 2, wherein each of the two arms include a width of at least about 2.5 cm.

5. The fluid collection device of claim 2, wherein each of the two arms include a width of at least about 5.1 cm.

6. The fluid collection device of claim 4, wherein the width of each of the two arms tapers towards the fluid collection device or the conduit.

7. The fluid collection device of claim 2, wherein each of the two arms includes a non-adhesive surface opposite to the adhesive surface.

8. The fluid collection device of claim 7, further comprising a removable cover on the adhesive surface of each of the two arms.

9. The fluid collection device of claim 2, wherein the two arms are detachably secured or securable to the fluid impermeable barrier in a position selected by the user or the caregiver.

10. The fluid collection device of claim 9, wherein each of the two arms extend at least about 2.5 cm from the fluid impermeable barrier.

11. The fluid collection device of claim 9, wherein each of the two arms extend at least about 5.1 cm from the fluid impermeable barrier.

12. The fluid collection device of claim 9, wherein the two arms are positioned or positionable less than about 7.6 cm from the aperture on the fluid impermeable barrier.

13. The fluid collection device of claim 9, wherein the two arms are positioned or positionable less than about 5.1 cm from the aperture on the fluid impermeable barrier.

14. The fluid collection device of claim 9, wherein the two arms are positioned or positionable less than about 2.5 cm from the aperture on the fluid impermeable barrier.

15. The fluid collection device of claim 9, wherein the two arms are secured to the fluid impermeable barrier with an adhesive.

16. (canceled)

17. (canceled)

18. The fluid collection device of claim 9, further comprising a ring configured to friction fit around the fluid impermeable barrier, wherein the two arms are secured to the ring.

19. The fluid collection device of claim 2, further comprising the conduit extending through the aperture such that a first portion of the conduit is disposed within the chamber and a second portion of the conduit is disposed outside the chamber, wherein the two arms are secured or securable to the second portion of the conduit.

20. The fluid collection device of claim 19, wherein each of the two arms extend at least about 2.5 cm from the second portion of the conduit.

21. The fluid collection device of claim 19, wherein each of the two arms extend at least about 5.1 cm from the second portion of the conduit.

22. The fluid collection device of claim 19, wherein the two arms are secured to the second portion of the conduit with an adhesive.

23. The fluid collection device of claim 19, wherein the two arms are ultransonically bonded to the second portion of the conduit.

24. The fluid collection device of claim 19, wherein the two arms are molded to the second portion of the conduit.

25. The fluid collection device of claim 19, further comprising a ring friction fit around the second portion of the conduit, wherein the two arms are secured to the ring.

26. A method of collecting fluids from a user, the method comprising:
positioning a fluid permeable body of a fluid collection device to cover a urethra of a user;
applying one or more adhesive arms to at least one of a pubic region, a pelvic region, or an abdominal region of the user effective to secure the fluid collection device to the user, the one or more adhesive arms being one of: (1) detachably secured or securable to a fluid impermeable barrier of the fluid collection device in a position selected by the user or a caregiver or (2) secured or securable to a conduit of the fluid collection device extending through an aperture in the fluid impermeable barrier, and
receiving fluids discharged from the urethra into a chamber of the fluid collection device.

27. The method of claim 26, wherein applying one or more adhesive arms to at least one of a pubic region, a pelvic region, or an abdominal region of the user effective to secure the fluid collection device to the user includes applying the one or more adhesive arms to the pelvic region of the user, the one or more adhesive arms being secured to the fluid impermeable barrier distal to an opening in the fluid impermeable barrier through which the fluids are collected from the urethra.

28. The method of claim 27, further comprising securing the one or more arms to the fluid impermeable barrier.

29. The method of claim 28, wherein securing the one or more arms to the fluid impermeable barrier includes friction fitting a ring on the fluid impermeable barrier, the one or more arms being secured to the ring.

30. The method of claim 26, wherein applying one or more adhesive arms to at least one of a pubic region, a pelvic region, or an abdominal region of the user effective to secure the fluid collection device to the user includes applying the one or more adhesive arms to the abdominal region of the user, the one or more adhesive arms being secured to the conduit that is in fluid communication with the chamber.

31. The method of claim 30, further comprising securing the one or more arms to the conduit.

32. The method of claim 31, wherein securing the one or more arms to the conduit includes fitting a ring on the conduit, the one or more arms being secured to the ring.
